(19) United States
(12) Patent Application Publication
(10) Pub. No.: US 2025/0241703 A1
(43) Pub. Date: Jul. 31, 2025

(54) ELECTROPORATION THERAPY FOR TURBINATE REDUCTION

(71) Applicant: Mayo Foundation for Medical Education and Research, Rochester, MN (US)

(72) Inventors: Jason A. Tri, Rochester, MN (US); Samuel J. Asirvatham, Rochester, MN (US); Jeffrey Tri, Rochester, MN (US)

(21) Appl. No.: 18/705,260

(22) PCT Filed: Oct. 14, 2022
(86) PCT No.: PCT/US2022/046710

§ 371 Date: Apr. 26, 2024

Related U.S. Application Data
(60) Provisional application No. 63/271,825, filed on Oct. 26, 2021.

(51) Int. Cl. A61B 18/14; A61B 18/00

(52) U.S. Cl. CPC A61B 18/1485; A61B 2018/00238; A61B 2018/00327; A61B 2018/00577; A61B 2018/00875; A61B 2018/1467

(57) ABSTRACT

Devices, systems, and methods described in this disclosure can be used to deliver electroporation to treat enlarged turbinate's and/or chronic sinusitis via the usage of electrical energy. For example, this document describes devices, systems, and methods for delivering thermal or non-thermal electroporation to treat enlarged turbinates and chronic sinusitis. Such an electroporation delivery device may optionally include one or more of the following features. The balloon may be made of a porous material. The one or more electrode spines may include four electrode spines. The one or more electrodes may include at least four electrodes. The balloon may be an elliptical, frustoconical, or conical shape when inflated. The electroporation therapy devices, systems, and methods described herein address limitations related to the current techniques for turbinate reduction (i.e., electrocautery, cryosurgery, or surgical reduction).

### CROSS-REFERENCE TO RELATED APPLICATIONS

This application claims the benefit of U.S. Provisional Application Ser. No. 63/271,825, filed Oct. 26, 2021. The disclosure of the prior application is considered part of (and is incorporated by reference in) the disclosure of this application.

### BACKGROUND

### 1. Technical Field

This document relates to devices and systems for delivering electroporation therapy and methods for their use. For example, this document relates to devices, systems, and methods for delivering electroporation to treat enlarged turbinates to mitigate nasal respiratory obstructions.

### 2. Background Information

Turbinates are small mucosa-covered structures inside the nose that cleanse and humidify air that passes through the nostrils and then into the lungs. Turbinates are bony structures surrounded by vascular tissue and a mucous membrane.

In some cases, turbinates can become swollen and inflamed by allergies, irritation, or infections. Such situations can cause nasal obstruction and produce an excessive amount of mucous that leads to congestion and further obstruction.

Nasal function is an important entrance that is used by the respiratory system, and it has been reported that 25% of the population suffers from non-allergenic nasal obstruction. Obstruction is most commonly caused by hypertrophy of the turbinates, but more specifically the inferior turbinates. Current techniques for turbinate reduction methods include electrocautery, cryosurgery, and surgical reduction. Each technique has its limitations.

### SUMMARY

This document describes devices and systems for delivering electroporation therapy and methods for their use. For example, this document describes devices, systems, and methods for delivering thermal or non-thermal electroporation to treat enlarged turbinates and chronic sinusitis. The thermal or non-thermal electroporation therapy devices, systems, and methods described herein address limitations related to the current techniques for turbinate reduction (i.e., electrocautery, cryosurgery, or surgical reduction).

In one implementation, an electroporation delivery device includes a shaft, a balloon attached to a distal end of the shaft, and one or more electrode spines attached to the balloon. Each of the one or more electrode spines includes one or more electrodes.

Such an electroporation delivery device may optionally include one or more of the following features. The balloon may be made of a porous material. The one or more electrode spines may include four electrode spines. The one or more electrodes may include at least four electrodes. The balloon may be an elliptical, frustoconical, or conical shape when inflated.

In another aspect, this disclosure is directed to an electroporation delivery device that includes a shaft, a bifurcated working end attached to a distal end of the shaft, and one or more electrodes attached to each of the arms. The bifurcated working end may include two arms.

Such an electroporation delivery device may optionally include one or more of the following features. Each arm of the two arms may be individually malleable into desired shapes. The one or more electrodes may each include four or more electrodes.

In another aspect, this disclosure is directed to a method of treating enlarged turbinates of a patient. The method includes advancing any of the electroporation delivery devices described herein into one or more nostrils of the patient, and delivering electroporation energy from the one or more electrodes to one or more of the turbinates of the patient.

Particular embodiments of the subject matter described in this document can be implemented to realize one or more of the following advantages. In some embodiments, the devices described herein are adjustable to accommodate various anatomic shapes and aspects of each nasal cavity.

Second, in some embodiments the devices and systems can be used to create a broad range of ablation lesion sets that are best suited for the individual patient.

Third, in some embodiments the devices and systems described herein can deliver a pre-pulse that may serve to numb the nerves, allowing for a painless application of energy to the tissue.

Fourth, in some embodiments the devices and systems described herein can perform thermal and/or non-thermal energy-based ablations (e.g., RF or pulsed electric field ablations). Through its versatile and adaptive design, the use of the electroporation therapy catheter devices described herein have the potential to significantly enhance the efficacy and ease of performing chronic sinusitis treatments and/or turbinate reduction.

Fifth, in some embodiments electroporation to treat enlarged turbinates and/or chronic sinusitis can be delivered in a minimally invasive fashion using the devices and methods provided herein. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 illustrates a first example electroporation device in accordance with some embodiments.

FIG. 2 illustrates a second example electroporation device in accordance with some embodiments.

Like reference numbers represent corresponding parts throughout.

### DETAILED DESCRIPTION

This document describes devices and systems for delivering electroporation therapy and methods for their use. For example, this document describes devices, systems, and methods for delivering thermal or non-thermal electroporation to treat enlarged turbinates and/or chronic sinusitis. The thermal or non-thermal electroporation therapy devices, systems, and methods described herein address limitations related to the current techniques for turbinate reduction (i.e., electrocautery, cryosurgery, or surgical reduction).

Electroporation can induce transfection of cells using a variety of vectors. It has also been used in oncology for the purpose of cell-specific destruction (e.g., of tumor cells). In accordance with the inventive concepts described herein, the utility of electroporation techniques lies in the potential for cell-specificity. For example, when a voltage is applied to a specific cellular milieu, the phospholipid bilayer of the cell permeabilizes depending on the size of the electric field to which it is exposed. Different cells have different bilayer components, thus resulting in differing electric field thresholds in terms of the size of the electric field required to induce a certain degree of membrane permeabilization. The larger the electric field, the more likely a cell membrane is to permeabilize to such an extent as to overcome the cell's intrinsic ability to repair the membrane. Accordingly, electroporation can be categorized into two approaches: reversible electroporation (which does not have the goal of cell death but the goal of cell membrane permeabilization for the purpose of delivery of specific vectors, drugs, etc.) and irreversible electroporation (which has the goal of cell death achieved by sufficient membrane permeabilization as to initiate the apoptosis cascade).

The inventors have discovered turbinate reduction electroporation devices and methods that, even at energy levels required to induce cell death, the energy levels can be controlled so that there is no effect on surrounding structures such as arteries, nerves, other tissues, and the like.

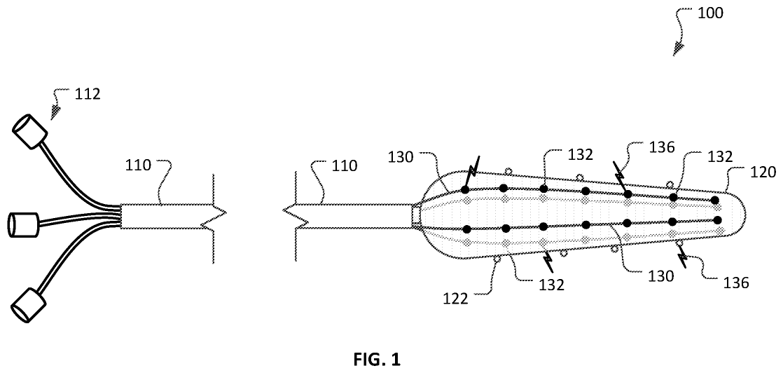

Referring to FIG. 1, a first example sinusitis treatment or turbinate reduction electroporation device 100 (or simply “electroporation device 100”) is depicted. The electroporation device 100 includes a shaft 110, a balloon 120, and electrode spines 130. The balloon 120 is attached to a distal end portion of the shaft 110. The electrode spines 130 are attached to the balloon 120.

The electroporation device 100 can be maneuvered into position to allow for ablation of chronic sinusitis or reduction of turbinate tissue. The balloon 120 can be inflated (e.g., using media such as saline, air, and the like). Radiofrequency or pulsed electric field ablation can then be delivered via the electrode spines 130. In some embodiments, the electroporation device 100 can be suitable for use with an endoscope for direct visualization of the placement of the balloon 120.

The shaft 110 carries the electrical wires for the electrode spines 130. In addition, the shaft 110 defines one or more lumens for transmitting the balloon inflation medium to and/or from the balloon 120. One or more connectors 112 can be located at the proximal end portion of the shaft 110. The one or more connectors 112 can be used to connect the electroporation device 100 to a control source of electroporation energy, to an inflation medium source, and the like.

The balloon 120 is inflatable and deflatable (by supplying an inflation medium to inflate the balloon 120, and by extracting the inflation medium to deflate the balloon 120). The balloon 120 is depicted in its inflated state in FIG. 1. In some embodiments, the balloon 120 is made of a compliant, flexible balloon material (e.g., silicone).

In some embodiments, the balloon 120 is made of a porous or micro-porous material. Accordingly, the inflation medium may elude from the interior of the balloon 120 to the surface of the balloon 120 (as represented by the surface droplets 122). In some embodiments, the inflation medium can be a conductive liquid like saline that readily conducts electricity. In such a case, the surface droplets 122 can conduct the energy from the electrodes of the electrode spines 130. This arrangement can help transmit the radiofrequency or pulsed electric field ablation energy from the electrodes to the target tissue. In some embodiments, one or more electrodes can additionally be located in the interior of the balloon 120 where the one or more electrodes are in direct contact with the inflation medium within the interior of the balloon 120.

In the depicted embodiment, the balloon 120 has a frustoconical shape when enlarged/inflated. Alternatively, the balloon 120 can be made to have various other shapes when enlarged/inflated. In some embodiments, the shape of the balloon 120 when enlarged/inflated is cylindrical, elliptical, and the like, without limitation. In the depicted embodiment, the longitudinal axis of the balloon 120 is coincident with the longitudinal axis of the shaft 110. In some embodiments, the longitudinal axis of the balloon 120 is partially or fully offset (e.g., angled) from the longitudinal axis of the shaft 110. In some embodiments, longitudinal axis of the balloon 120 is curved.

The electroporation device 100 also includes the electrode spines 130. In the depicted embodiment, four of the electrode spines 130 are included. In some embodiments, one, two, three, five, six, seven, eight, or more than eight of the electrode spines 130 are included. In some embodiments, each electrode spine 130 can be independently energized.

The electroporation energy delivered by the electrode spines 130 can be omnipolar, monopolar or bipolar. For example, one or more of the electrode spines 130 can operate as the anode and the other one or more of the electrode spines 130 can operate as the cathode in some embodiments. Such arrangements can be selectively configurable and controllable by the user. In some embodiments, all of the electrode spines 130 can operate as cathodes and a return electrode (e.g., a skin patch) can be used.

Each electrode spine of the electrode spines 130 includes one or more electrodes 132. In some embodiments, when there are two or more electrodes 132 on an electrode spine 130, each of the two or more electrodes 132 can be independently energized. In some embodiments, when there are two or more electrodes 132 on an electrode spine 130, each of the two or more electrodes 132 are energized as a single unit.

In some embodiments, for treating chronic sinusitis or reduction of turbinate tissue the pulse duration energy delivered to the tissue from the electrodes 132 will range from 50 nanoseconds up to 100 microseconds, and will have a voltage of 500 volts DC to 15 kilovolts DC.

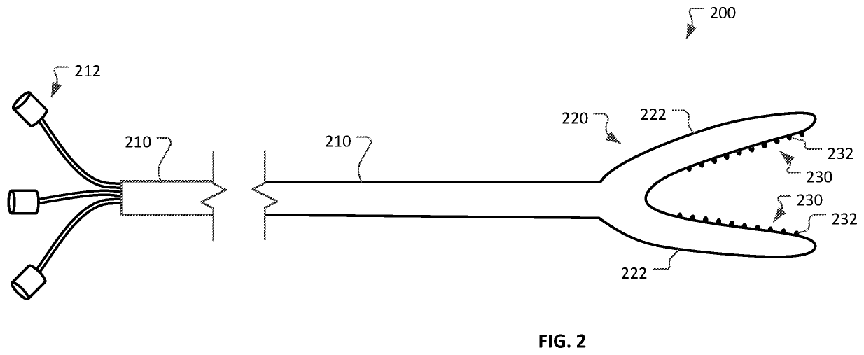

Referring to FIG. 2, a second turbinate reduction electroporation device 200 (or simply “electroporation device 200”) is depicted. The electroporation device 200 includes a shaft 210, a bifurcated working end 220, and electrodes 230. The working end 220 is attached to a distal end portion of the shaft 210. The electrodes 230 are attached to the working end 220.

The electroporation device 200 can be maneuvered into the nostrils to deliver electroporation for reduction of turbinate tissue. The bifurcated working end 220 can be shaped/formed into a desired configuration as desired to match a particular individual patient's anatomy. Radiofrequency or pulsed electric field ablation can then be delivered to the inferior turbinates via the electrodes 230.

The shaft 210 carries the electrical wires for the electrodes 230. One or more connectors 212 can be located at the proximal end portion of the shaft 210. The one or more connectors 212 can be used to connect the electroporation device 100 to a control source of electroporation energy, and the like.

The bifurcated working end 220 includes two arms 222 (one for each nostril). Each of the arms 222 is malleable, moldable, and adjustable to allow a user to shape the arms 222 to match a particular individual patient's anatomy. The arms 222 maintain their shapes after being manipulated by the user.

The electrodes 230 are disposed on the two arms 222 and are positioned to contact the inferior turbinates when the bifurcated working end 220 is placed within the nostrils of a patient. The electrodes 230 include one or more individual electrodes 232.

The electroporation energy delivered by the electrodes 232 can be omnipolar, monopolar or bipolar. For example, one or more of the electrodes 232 can operate as the anode and the other one or more of the electrodes 232 can operate as the cathode in some embodiments. Such arrangements can be selectively configurable and controllable by the user. In some embodiments, all of the electrodes 232 can operate as cathodes and a return electrode (e.g., a skin patch) can be used.

In some embodiments, when there are two or more electrodes 232 on an arm 222, each of the two or more electrodes 232 can be independently energized. In some embodiments, when there are two or more electrodes 232 on an arm 222, each of the two or more electrodes 232 are energized as a single unit.

In addition, in some embodiments the electroporation device 200 has the option for adjustment of electrode size, orientation, and separation of electrodes 232.

In some embodiments, for reduction of turbinate tissue the pulse duration energy delivered to the tissue from the electrodes 232 will range from 50 nanoseconds up to 100 microseconds, and will have a voltage of 500 volts DC to 15 kilovolts DC.

### Additional Optional Embodiments, Features and Uses

In another embodiment, a device is configured to allow for a non-invasive application of energy. Such a device places an electrode longitudinally along the nose over the sinus. The return electrode is then be placed on the opposite side in the same configuration. Energy will be delivered across the nasal cavities along for a single application to remove all unwanted tissue.

While in the depicted embodiments the electrodes are DC electrodes, alternatively, or additionally, some embodiments of intravascular electroporation catheters can be configured to deliver other types of electroporation energy such as, but not limited to, radiofrequency (RF), AC, cryogenic, chemical, and the like. In some embodiments, a combination of such energy sources can be used within a single embodiment of intravascular electroporation catheter (e.g., RF and DC are used in combination is some embodiments). The electroporation energy can be omnipolar, monopolar or bipolar. In some implementations, two or more types of electroporation energy sources can be coupled to electrodes.

In some embodiments, the devices described herein are adjustable to accommodate various anatomic shapes and aspects of each nasal cavity.

In some embodiments, the devices and systems can be used to create a broad range of ablation lesion sets that are best suited for the individual patient.

In some embodiments, the devices and systems described herein can deliver a pre-pulse that may serve to numb the nerves, allowing for a painless application of energy to the tissue.

To facilitate minimally-invasive electroporation of the terminates, in some embodiments, a large surface area cathode is placed external to the nostrils and the sinuses with the anode being a very fine (e.g., 0.32 mm or less) wire(s) that are placed into the nasal cavity. By varying the surface electrode or surface area used, the electroporation field can be focused on the turbinates (including the inferior turbinates) without the discomfort of placing larger elements into the nasal cavity. Potentially, such iterations can be used at home by patients for slow, gradual reduction in the turbinates. The anode and cathode can be interchanged in this and other embodiments described herein.

In order to vary the surface area of the surface electrode (anode or cathode), some embodiments include a variable adapter electrode system. One structure to achieve this is an adjustable iris-like insulation sleeve on the electrode, with the iris aperture having an opening size that can be modulated with a dial or other element, and test pulses given at various aperture sizes with impedance measured from the intra-nostril electrode or another surface electrode to know if the field intensity is appropriate at the site of interest, i.e., the inferior turbinate for example. In another structure, the surface electrode is made up of multiple, smaller electrodes separated by an insulating material. Various electroporation energy vectors can be selectively created based on which of the electrodes is/are being used as the surface electrode (anode or cathode).

In another embodiment for noninvasive delivery of electroporation to the turbinate(s), the intra-nasal “electrode” instead of being a wire can be a nasal spray with ionized metallic material that can serve as an aerosolized electrode. This embodiment would be of particular value for patients who are sensitive to having an element placed in the nostril, or where more widespread mucosal electroporation (such as for recurrent sinusitis, mucositis, rhinitis, medicamentosus, etc.) is the directed goal (disease treated) for the electroporation therapy.

One of the advantages of electroporation as described herein, and a key part of the systems and methods described herein, is to prevent thermal damage or edema. However, electroporation energy by itself may produce thermal effects at high doses, etc. In order to titrate/modulate the appropriate amount of energy delivered/required, some embodiments include a feedback system in which the mucosally-placed electrodes can be used to provide local electrical signals from the tissues being electroporated. In some embodiments, the use of these electrical signals will allow appropriate limiting of the electroporation energy and sequence (including in an automated fashion in some embodiments).

In some embodiments, the inter-electrode impedance is measured. The impedance would be expected to fall with appropriate electroporation as has been demonstrated in other tissues. Once a plateau with this fall is achieved, the electroporation is automatically or manually reduced/titrated to hold at that level and prevent the possibility of collateral injury or thermal effects.

In another embodiment for dose modulation/titration, the electrical impedance is used as a marker for tissue edema. Since tissue edema can produce worsening of patient symptoms in some cases, further decrease in impedance from the edema can be monitored with real-time impedance being used for electroporation feedback, as well as more peripheral electrodes in some embodiments. Thus, if the primary electrode(s) show an initial impedance change as a result of electroporation, but subsequently peripheral electrodes pick up a new further change in impedance, then edema is likely occurring, and the electroporation energy delivery would be automatically turned off.

In some embodiments that include real-time electroporation energy modulation/titration, infrared or other light-based measures including photoplethysmography are used so as to monitor for edema and/or collateral tissue destruction.

Although turbinate hypertrophy is the primary goal of the systems and methods described in this disclosure, treatment of tumors, polyps, recurrent sinusitis, allergic mucositis, vasomotor mucositis, etc., could all be targeted with the devices and techniques described herein for energy delivery. Similarly, infectious processes including viral rhinitis (such as with the rhinovirus, coronavirus, or other viruses) can be treated at source periodically with the devices and techniques described herein, particularly in patients who are prone for recurrent mucositis (a potential risk factor for more severe viral infections).

The systems and methods described herein may also be used to deliver medications that can be antiinflammatory or antihypertrophic (such as steroid preparations, antimycotic agents, and the like). These agents may have toxic side effects when administered or absorbed into the systemic circulation. The use of electroporation to target delivery where the medications are needed without excessive systemic absorption will be facilitated by these systems and methods.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. An electroporation delivery device comprising:
a shaft;
a balloon attached to a distal end of the shaft; and
one or more electrode spines attached to the balloon, each of the one or more electrode spines including one or more electrodes.

2. The electroporation delivery device of claim 1, wherein the balloon is made of a porous material.

3. The electroporation delivery device of claim 1, wherein the one or more electrode spines includes four electrode spines.

4. The electroporation delivery device of claim 1, wherein the one or more electrodes includes at least four electrodes.

5. The electroporation delivery device of claim 1, wherein the balloon is an elliptical, frustoconical, or conical shape when inflated.

6. An electroporation delivery device comprising:
a shaft;
a bifurcated working end attached to a distal end of the shaft, the bifurcated working end comprising two arms; and
one or more electrodes attached to each of the arms.

7. The electroporation delivery device of claim 6, wherein each arm of the two arms are individually malleable into desired shapes.

8. The electroporation delivery device of claim 6, wherein the one or more electrodes each comprises four or more electrodes.

9. A method of treating enlarged turbinates of a patient, the method comprising:
advancing the electroporation delivery device of any one of claims 1 through 8 into one or more nostrils of the patient; and
delivering electroporation energy from the one or more electrodes to one or more of the turbinates of the patient
